(19) United States
(12) Patent Application Publication
(10) Pub. No.: US 2003/0078539 A1
(43) Pub. Date: Apr. 24, 2003

(54) Perfusion catheter and membrane

(76) Inventors: Eric D. Peterson, Fremont, CA (US); Jessica Chiu, Belmont, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 09/749,708

(22) Filed: Dec. 27, 2000

(51) Int. Cl. A61M 29/00; A61M 31/00; A61M 37/00

(52) U.S. Cl. 604/103.01; 604/103.02; 604/103.08

(57) ABSTRACT

A catheter having a perfusion section encapsulated by a porous membrane. The catheter can include a shaft to accommodate a radiation source. A spiraled balloon can be positioned about the shaft for centering within a body lumen. Additionally, a longitudinal balloon or mechanical expansion can be provided about the shaft for positioning within a body lumen. A radiotherapy system can be included with a radiation source and a catheter having a perfusion section encapsulated by a porous membrane. A method is provided where a catheter with a perfusion section encapsulated by a porous membrane is advanced through a body lumen. The body lumen is treated by the catheter while a body fluid is perfused past the perfusion section.

### BACKGROUND OF THE INVENTION

The present invention relates to intravascular therapy. In particular, the present invention relates to a perfusion catheter.

### BACKGROUND OF THE PRIOR ART

For patients with arterial blockage, such as coronary or peripheral stenotic lesions, angioplasty is often used. Through angioplasty, an angioplasty catheter is delivered to a vessel region which has been narrowed by a stenosis (atherosclerotic plaque build-up). A balloon of the angioplasty catheter is inflated to compress the stenosis against the vessel wall. The balloon is then deflated and the angioplasty catheter removed. This procedure widens an inner diameter of the arterial lumen, allowing for increased blood flow through the vessel region formerly narrowed by the stenosis. Often, a stent is placed at the vessel region in conjunction with the angioplasty procedure.

However, restenosis, in which the senotic narrowing of the vessel returns, is common after angioplasty procedures. To prevent restenosis, radiotherapy procedures are used to impair cell growth by exposing potential restenosis sites to radiation.

To deliver radiation to the potential restenosis sites, a variety of radiotherapy procedures may be used. Source wire radiotherapy, for example, utilizes a source wire positioned by a radiotherapy catheter. A balloon rests about a shaft of the radiotherapy catheter at a distal portion thereof. The balloon is inflated once it has been positioned adjacent the site of a former stenosis. A source wire, having a radioactive distal tip, is then advanced through the catheter to the aforementioned distal portion of the shaft, where radiation is emitted from the radioactive distal tip toward the site of the former stenosis. After a predetermined radiation exposure time, the source wire is retracted, the balloon deflated and the radiotherapy catheter removed.

During such a radiotherapy procedure a balloon shaped to avoid occlusion of the vessel being treated is often used. For example, if a longitudinal and cylindrically shaped balloon is inflated within the vessel, it will occlude blood flow during the radiotherapy procedure. The effects of such an occlusion reach beyond the particular vessel which is being treated. For example, a primary vessel being directly treated with the angioplasty balloon likely services other side branch vessels which branch out from the primary vessel. If the primary vessel is occluded proximal of (i.e., upstream), or in the area of, a side branch, the side branch can also be occluded as a direct result.

Where radiotherapy is used, it may result in vessel occlusions requiring parameters to be limited to short and intense radiotherapy treatments to limit ischemic episodes. Additionally, in order to alleviate an occlusion caused by the radiotherapy device, the radiotherapy may need to be periodically interrupted to allow perfusion of blood past the balloon in preventing ischemic episodes.

Alternatively, to avoid occlusions of the vessel during radiotherapy, the radiotherapy catheter may be designed to permit perfusion of blood past the balloon even where the balloon is inflated. Such “perfusion balloons” may include balloons which are multi-lobed or spiraled about a distal portion of the catheter shaft. In this way blood is allowed to perfuse past the balloon via channels between balloon lobes or between spiral threading of the balloon.

The perfusion channels of perfusion balloons, however, are susceptible to being blocked by vessel imperfections. That is, following an angioplasty procedure, it is unlikely that the vessel will include no more than a smooth tubular interior. Rather, the wall of the vessel will likely be rough and fairly non-uniform with obtrusive features likely present. These obtrusive vessel imperfections can interfere with perfusion of blood through a perfusion channel. A single obtrusive feature can potentially occlude all blood flow through a perfusion channel, even where the remainder of the channel remains un-occluded. Therefore, what is needed is a catheter configured to allow perfusion without allowing occlusion of a perfusion channel.

### SUMMARY OF THE INVENTION

An embodiment of the invention includes a catheter with a perfusion section and a membrane encapsulating a portion of the perfusion section. The membrane has pores.

Another embodiment of the invention includes a catheter with a shaft to accommodate a radiation source. A spiraled perfusion balloon to center the shaft within a body lumen is provided with a membrane encapsulating a portion of the balloon.

In another embodiment a catheter includes a shaft to accommodate a radiation source. A longitudinal balloon is included about the shaft for positioning within a body lumen. A membrane encapsulating a portion of the balloon is also provided.

In yet another embodiment a catheter includes a shaft to accommodate a radiation source. A mechanical expansion coupled to the shaft is provided to position the shaft within a body lumen. A membrane encapsulating a portion of the balloon is also provided.

Another embodiment of the invention includes a radiotherapy system with a radiation source. A catheter with a perfusion section and a membrane thereabout is also provided.

In a method of the invention a catheter is advanced through a body lumen. The catheter includes a perfusion section with a membrane. A portion of the body lumen is treated by the catheter while a body fluid is perfused past the perfusion section.

### BRIEF DESCRIPTION OF THE DRAWINGS

FIG. 1 is a pictorial view of an embodiment of the present invention.

FIG. 2 is a sectional view of a lobed embodiment of the present invention inserted within a vessel.

FIG. 3 is a front cross sectional view of an embodiment of the present invention, taken from line 3-3 of FIG. 2.

FIG. 4 is a side sectional view of an alternate embodiment of the present invention.

FIG. 5 is a side sectional view of an alternate embodiment of the present invention.

### DETAILED DESCRIPTION OF THE INVENTION

The following description makes reference to numerous specific details in order to provide a thorough understanding of the present invention. However, each and every specific detail need not be employed to practice the present invention. Additionally, well-known details, such as particular materials or methods, have not been described in order to avoid obscuring the present invention.

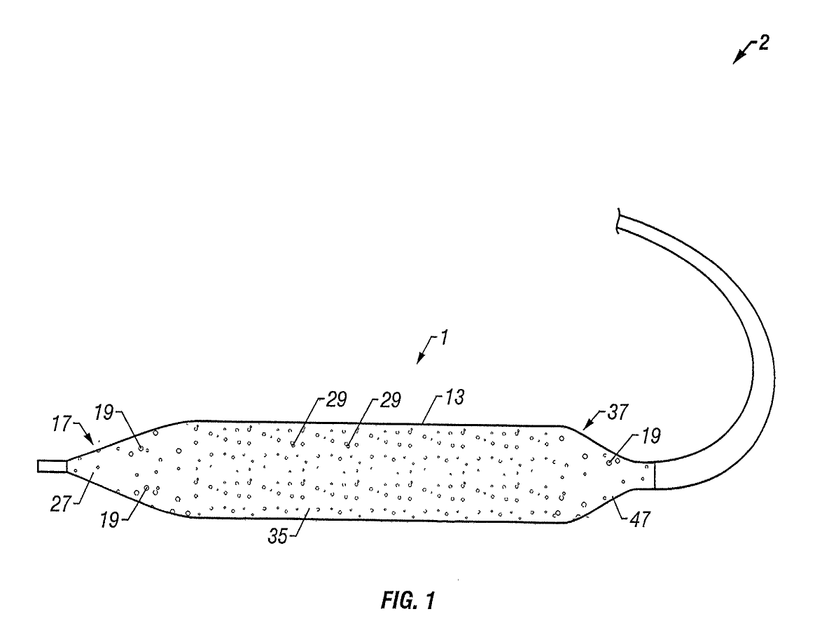

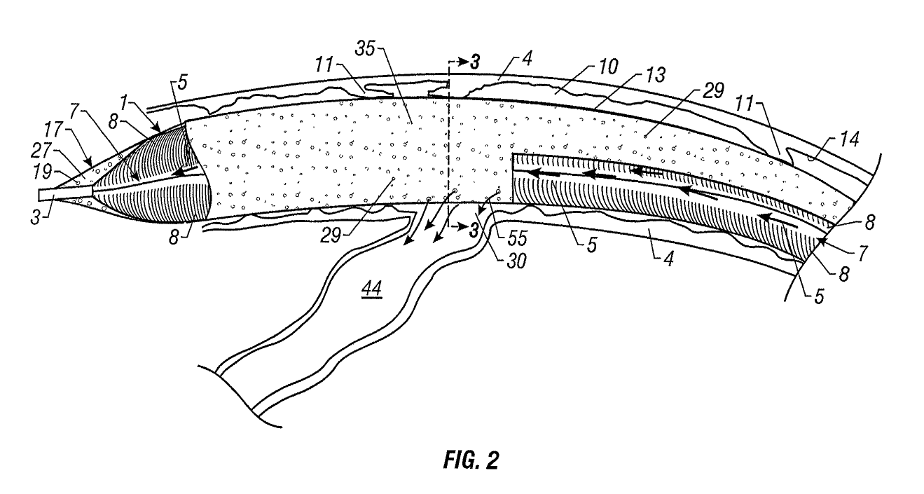

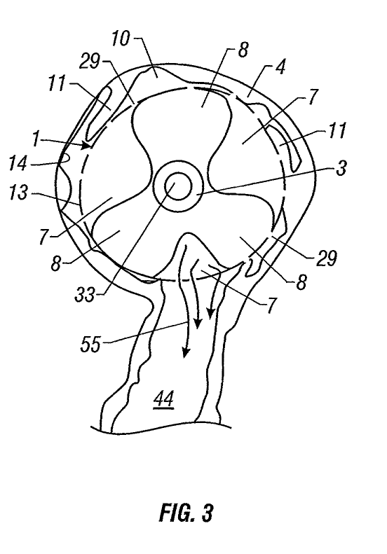

Referring to FIG. 1 an embodiment of a perfusion catheter 2 with a perfusion balloon 1 at a distal end thereof is shown. In FIG. 2 the perfusion balloon 1 is shown within a primary vessel 4 as partially sectioned to reveal interior features. FIG. 3 shows a cross sectional view of the perfusion balloon 1 of FIG. 2.

The perfusion balloon 1 of the embodiment shown is constructed to allow perfusion of a body fluid past a shaft 3. In the embodiment shown, the perfusion balloon 1 is also constructed to center the distal portion of the shaft 3 within a body lumen, such as the primary vessel 4. For example, in a method of the invention, centering of the distal portion of the shaft 3 is provided where the shaft 3 is to accommodate a source wire having a radioactive distal tip during a radiotherapy procedure. In this manner, an even distribution of radiation, emanating from the distal portion of the shaft 3, which houses the radioactive source wire via a source wire lumen 33, is delivered to the primary vessel 4 to be treated (see also FIG. 3).

The perfusion balloon 1 is configured to allow a primary flow of blood (arrows 5) to move past the perfusion balloon 1 even though the perfusion balloon 1 rests within the primary vessel 4. That is, the perfusion balloon 1 is configured to allow perfusion. The natural flow of blood through the primary vessel 4 of FIG. 2 would be from right to left as shown. In order to allow perfusion, the perfusion balloon 1 is equipped with perfusion channels 7 (also shown in FIG. 3). Each perfusion channel 7 is located between individual balloon lobes 8 of the perfusion balloon 1. The embodiment shown includes a perfusion balloon 1 having three lobes 8 (i.e., a “tri-lobed” balloon). Therefore, three perfusion channels 7, one between each lobe 8, are provided.

The perfusion balloon 1, having lobes 8, runs longitudinally along the shaft 3 throughout a perfusion section of the catheter. Perfusion channels 7 prevent the perfusion balloon 1 from resting flush against the vessel wall 14 which could occlude the primary flow of blood (arrows 5). As a result, the perfusion balloon 1 can remain within a lumen 10 of the primary vessel 4 for a significant period of time without inducing an ischemic condition. For example, due to the perfusion channels 7, the perfusion balloon 1 can remain within the primary vessel 4 throughout the duration of a radiotherapy procedure without obstructing the primary flow of blood (arrows 5).

While the lobes 8 and perfusion channels 7 avoid complete occlusion of the lumen 10, the perfusion channels 7 would be susceptible to partial blockage by irregularities 11 present in the vessel wall 14 if not for a membrane 13 discussed further below. That is, the vessel wall 14 is not generally smooth. This is especially true in portions of the primary vessel 4 which have experienced injury, such as the site of a former stenotic lesion which is likely to have thrombus projections. So, for example, if the perfusion balloon 1 is being used during a radiotherapy procedure following PTCA, there is a high probability that the vessel wall 14 includes a large number of irregularities 11 in the area that is being treated (i.e. adjacent the perfusion balloon 1). These irregularities 11 often include flaps or other protruding shapes which can extend into a portion of a perfusion channel 7. Such a partial blockage of a perfusion channel 7 decreases the overall efficiency of perfusion.

In order to ensure effective perfusion, the perfusion balloon 1 is surrounded by a membrane 13. Once the catheter has been positioned, with the perfusion balloon 1 adjacent a vessel wall 14 having irregularities, the perfusion balloon 1 is inflated (as shown in FIG. 2). Upon inflation, the membrane 13 is forced against the vessel wall 14 preventing the irregularities 11 from protruding into the perfusion channels 7. The previously protruding irregularities 11 are forced to fold up against other portions of the vessel wall 14 by the expanded membrane 13. Thus, the irregularities are forced away from the perfusion channels 7 allowing the perfusion channels to remain open and unobstructed. A more efficient primary flow of blood (arrows 5) is maintained.

The membrane 13 is expandable and responsive to the inflatable characteristics of the perfusion balloon 1. When the lobes 8 are inflated, the membrane 13 expands outward. In the embodiment shown, the membrane 13 is made of an elastic biocompatible material such as polysiloxane or polysiloxane related substances or derivatives capable of expansion.

Of note is the fact that, while a membrane 13 is provided, it does not prevent the primary flow of blood (arrows 5) from entering or exiting the perfusion channels 7. Rather, blood is allowed access to the perfusion channels 7 at a proximal end 37 and an exit at the distal end 17 of the perfusion balloon 1.

In one embodiment, the entry and exit of the primary flow of blood (arrows 5) via perfusion channels 7 is provided by using a membrane 13 which surrounds the perfusion balloon 1 circumferentially only. Such a membrane 13 is secured directly to the perfusion balloon 1. Thus, the proximal end 37 and the distal end 17 of the perfusion balloon 1 would be left open allowing the perfusion channels 7 to be directly open to the lumen 10 of the primary vessel 4.

Alternatively, as shown in the embodiment of FIGS. 1 and 2, the membrane 13 is attached to the shaft 3 distal of the perfusion balloon 1 and proximal of the perfusion balloon 1. A distal portion 27 and a proximal portion 47 of the membrane 13 are equipped with access pores 19 to allow entry and exit of the primary flow of blood (arrows 5) through the perfusion channels 7. When the perfusion balloon 1 and the membrane 13 are in an expanded state the access pores 19 have a diameter of between about 0.01 and about 0.05 inches and occupy between about 10 and about 50 percent of the surface area of the membrane 13 at its proximal and distal portion 27. In one embodiment the access pores have a diameter of about 0.028 inches and occupy about 25 percent of the surface area of the membrane 13 in an expanded state. In one embodiment, the membrane 13 is securely attached to the shaft 3 and circumferentially surrounds the perfusion balloon 1 without inhibiting perfusion. This optimizes security of the membrane 13 and ensures that no portion of the perfusion channel 7 is susceptible to occlusion by protruding irregularities 11.

Referring specifically to FIG. 2, while the membrane 13 is configured to avoid inhibition of perfusion through the primary vessel 4, a side branch vessel 44 branches off of the primary vessel 4. Ideally, the side branch vessel 44 also remains un-occluded. An un-occluded side branch vessel 44 would require a side branch blood flow (arrows 55) emanating from the primary flow of blood (arrows 5). However, as shown, the portion of the primary vessel 4 being treated with the perfusion balloon 1 includes an intersection with a side branch vessel 44. Therefore, the perfusion balloon 1 and membrane 13 rest across the entryway 30 to the side branch vessel 44.

In order to prevent occlusion of the side branch vessel 44 by the membrane 13, the membrane 13 is equipped with perfusion pores 29. That is, the primary flow of blood (arrows 5) through the perfusion channels 7 is able to exit corresponding perfusion channels 7 at the entryway 30 through the perfusion pores 29 as side branch blood flow (arrows 55). The embodiment shown has perfusion pores 29 throughout the body 35 of the membrane 13 to ensure that the side branch vessel 44 is not occluded by the body 35 of the membrane 13.

As shown in the embodiment of FIG. 2, access pores 19 are provided to ensure a continued primary flow of blood (arrows 5) past the perfusion balloon 1. Likewise, the perfusion pores 29 are provided to ensure a continued side branch blood flow (arrows 55). The perfusion pores 29 take up between about 10 and about 50 percent of the surface area of the body 35 of the membrane 13. The perfusion pores 29 are between about 0.01 and about 0.05 inches in diameter when the perfusion balloon 1 and the membrane 13 are in an expanded state (as shown in FIG. 2). In one embodiment the perfusion pores 29 take up about 25 percent of the surface area of the body 35 and are about 0.014 inches in diameter when the perfusion balloon 1 is expanded.

Referring to FIG. 3, a cross section taken from line 3-3 of FIG. 2 is shown. From a new perspective, the perfusion balloon 1 is shown within a lumen 10 of the primary vessel 4. All three perfusion channels 7 can be seen between the three lobes 8. Again, the lobes 8 are provided about a shaft 3 and surrounded by the membrane 13. The shaft 3 is equipped with a now visible source wire lumen 33 to accommodate a radiotherapy mechanism such as a source wire with a radioactive distal tip (not shown). In another embodiment the lumen 33 accommodates radiation pellets to deliver radiotherapy. In other embodiments additional forms of radiotherapy are provided via the shaft 3 and perfusion balloon 1.

In FIG. 3, the extent to which the membrane 13 holds irregularities 11 against other portions of the vessel wall 14 and away from the perfusion channels 7 leaving the perfusion channels 7 un-occluded can be seen. While keeping irregularities 11 from occluding the perfusion channels 7, the membrane 13 also accounts for the intersection of a side branch vessel 44. That is, perfusion pores 29 have been provided in the membrane 13 which allow a side branch blood flow (arrows 55) to cross the membrane from a perfusion channel 7 and into the side branch vessel 44. Thus, the membrane 13 has prevented occlusion of the perfusion channels 7 without causing occlusion of the side branch vessel 44.

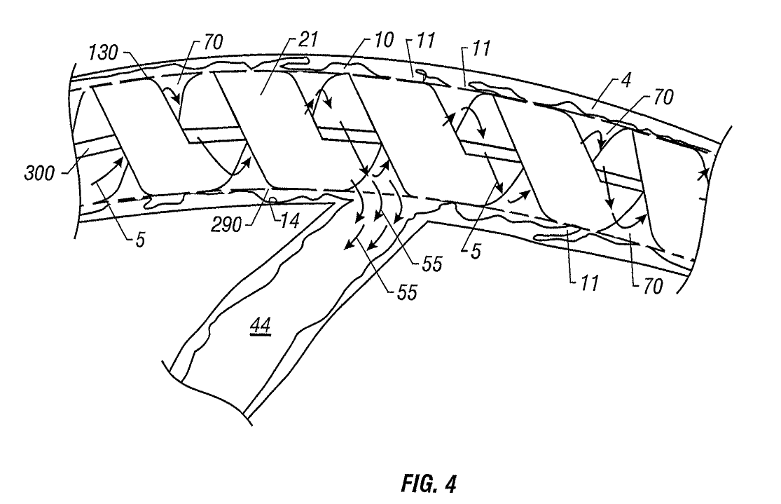

Referring to FIG. 4 an alternate embodiment of the invention is shown, again making use of a perfusion catheter. Likewise, the primary vessel 4 having irregularities 11 is to be treated. Where treatment includes radiation delivery, the catheter also provides centering capability for delivery of a uniform level of radiation from a shaft 300. During such a treatment irregularities 11 are restrained from interfering with features of the adjacent catheter. A side branch vessel 44 is shown intersecting the primary vessel 4 in an area where the primary vessel 4 is accommodating the catheter. Therefore, embodiments of the invention are able to restrain the irregularities 11 in a manner which does not cause occlusion of the side branch vessel 44.

In order to force the irregularities against the primary vessel wall 14, a membrane 130 is provided surrounding a perfusion section of the catheter. The membrane 130 is configured to allow a side branch blood flow (arrows 55) to the side branch vessel 44. Thus, perfusion pores 290 are provided in the membrane 130.

The perfusion section of the catheter includes a spiraled perfusion balloon 21 about the shaft 300 of the catheter. The spiraled perfusion balloon 21 provides a perfusion channel 70 between adjacent threads of the spiraled perfusion balloon 21. The perfusion channel 70 allows perfusion through the primary vessel 4. The spiraled perfusion balloon 21 is a balloon which spirals around the shaft 300. By spiraling around the shaft 300 interaction between the spiraled perfusion balloon 21 and the membrane 130 is maximized. The spiral shape of the spiraled perfusion balloon 21 forces the membrane 130 open in a circumferential manner. The spiraled perfusion balloon 21 is therefore, particularly adept at keeping the membrane 130 circumferentially expanded against the primary vessel wall 14.

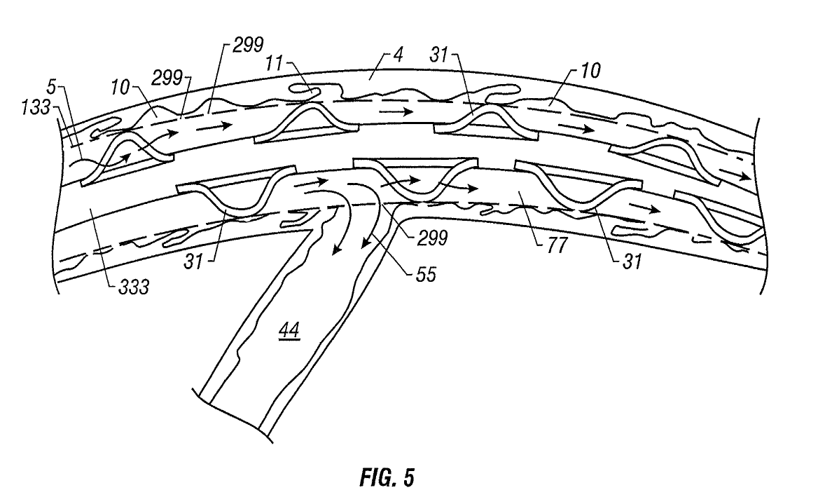

Referring to FIG. 5 another embodiment of the invention is shown. Again, a perfusion catheter is provided within a primary vessel 4 having irregularities 11. Where treatment includes radiation delivery, the catheter also provides centering capability for delivery of a uniform level of radiation from shaft 333.

In the embodiment of FIG. 5, the perfusion section of the catheter includes mechanical expansions 31 which arise from the shaft 333. The mechanical expansions 31 may be ribbons of metal, plastic, or other material designed to expand out into a hump-like shape to hold open the membrane 133 and the primary vessel 4. That is, rather than providing an inflatable balloon configured to allow a particularly shaped perfusion channel 77, a mechanical mechanism is provided to allow perfusion (and centering capability). The mechanical expansions 31 leave all other portions of the catheter free to allow a primary flow of blood (arrows 5) there through within the primary vessel 4. That is, the perfusion channel 77 is defined by the shaft 333 and the membrane 133 (where present) with the only interruption being the narrow mechanical expansions 31. This allows for efficient perfusion through the primary vessel 4 and to the side branch 44.

Embodiments of the present invention include a perfusion balloon with perfusion channels having an ability to avoid occlusion of the perfusion channels. Additionally, embodiments of the invention also include configurations that avoid occlusion of side branch vessels emanating from a more primary vessel being treated. Although an exemplary embodiment of the invention has been shown and described in the form of particular membranes with pores, many changes, modifications, and substitutions may be made by one having ordinary skill in the art without necessarily departing from the spirit and scope of this invention.

We claim:

1. A catheter comprising:
a perfusion section; and
a membrane encapsulating a portion of said perfusion section, said membrane having pores.

2. The catheter of claim 1 wherein said membrane is comprised of a polysiloxane related substance.

3. The catheter of claim 1 wherein said perfusion section includes at least one perfusion channel, a portion of said perfusion channel open to a body lumen when said catheter is inserted therein.

4. The catheter of claim 1 wherein said pores include perfusion pores of between about. 0.01 and about 0.05 inches in diameter when said membrane is in an expanded state.

5. The catheter of claim 1 wherein said pores have a concentration of between about 10% and about 50% of a surface area of said membrane when said membrane is in an expanded state.

6. The catheter of claim 1 wherein said membrane further comprises:
a body; and
an end portion adjacent said body, said end portion coupled to a shaft, said shaft extending through said perfusion section.

7. The catheter of claim 6 wherein said pores include access pores throughout said end portion.

8. The catheter of claim 7 wherein said access pores are between about 0.01 and about 0.05 inches in diameter when said membrane is in an expanded state.

9. The catheter of claim 1 further comprising:
a shaft through said perfusion section; and
a perfusion balloon about said shaft.

10. The catheter of claim 9 wherein said shaft includes a shaft lumen to accommodate a radiation source, said perfusion balloon to center said shaft within a body lumen when said catheter is inserted therein.

11. The catheter of claim 9 wherein said perfusion balloon further comprises at least one lobe and said perfusion section includes at least one perfusion channel adjacent said at least one lobe.

12. The catheter of claim 11 wherein said at least one lobe includes a spiraled lobe about said shaft.

13. The catheter of claim 11 wherein said at least one lobe includes a longitudinal lobe parallel said shaft.

14. The catheter of claim 1 further comprising:
a shaft through said perfusion section; and
at least one mechanical expansion coupled to said shaft.

15. The catheter of claim 14 wherein said shaft includes a shaft lumen to accommodate a radiation source, said at least one mechanical expansion to center said shaft within a body lumen when said catheter is inserted therein.

16. A catheter comprising:
a shaft with a lumen there through to accommodate a radiation source;
a spiraled perfusion balloon about said shaft to center said shaft within a body lumen; and
a membrane encapsulating a portion of said perfusion balloon, said membrane having pores.

17. A catheter comprising:
a shaft with a lumen there through to accommodate a radiation source;
at least one longitudinal perfusion balloon about said shaft to position said shaft within a body lumen; and
a membrane encapsulating a portion of said perfusion balloon, said membrane having pores.

18. A catheter comprising:
a shaft with a lumen there through to accommodate a radiation source;
at least one mechanical expansion coupled to said shaft to position said shaft within a body lumen; and
a membrane encapsulating a portion of said perfusion balloon, said membrane having pores.

19. A radiotherapy system comprising:
a radiation source; and
a catheter having a perfusion section with a membrane at least partially thereabout, said membrane having pores.

20. The radiotherapy system of claim 19 wherein said catheter includes a shaft through said perfusion section to accommodate said radiation source, said radiation source comprising a radioactive distal tip of a radiation source wire.

21. A method comprising:
advancing a catheter through a first body lumen, said catheter having a perfusion section with a membrane at least partially thereabout, said membrane having pores;
treating a portion of said first body lumen with a therapy provided by said catheter; and
perfusing a body fluid through said perfusion section during said treating

22. The method of claim 21 further comprising allowing said body fluid access to a second body lumen during said perfusing, said second body lumen intersecting said first body lumen at an opening, said membrane covering said opening.

23. The method of claim 21 wherein said therapy is radiotherapy provided through a shaft of said catheter, said shaft running through said perfusion section.
